(19) United States
(12) Patent Application Publication
(10) Pub. No.: US 2023/0166040 A1
(43) Pub. Date: Jun. 1, 2023

(54) CASSETTE AND MEDICAMENT DELIVERY DEVICE

(71) Applicant: SHL MEDICAL AG, ZUG (CH)

(72) Inventor: Ming-Ting Yin, Taipei City (TW)

(21) Appl. No.: 17/921,302

(22) PCT Filed: May 7, 2021
(86) PCT No.: PCT/EP2021/062127

§ 371 Date: Oct. 25, 2022

(30) Foreign Application Priority Data
May 27, 2020 (EP) 20176689.6

(51) Int. Cl. A61M 5/24; A61M 5/315; A61M 5/32

(52) U.S. Cl. CPC A61M 5/2466; A61M 5/31501; A61M 5/3205; A61M 5/3204

(57) ABSTRACT

A cassette is disclosed for receiving a medicament container, the cassette having a medicament delivery member; a guard for covering the medicament delivery member; a shell movable between a proximal shell position where movement of the guard is prevented by the shell, and a distal shell position where the guard is exposed from the shell; a biasing device arranged to force the shell towards the distal shell position; a blocking device movable from a blocking position where the shell is blocked from moving to the distal shell position, to a releasing position where the shell is released; and a locking device movable from a first position to a second position; wherein the blocking device and the locking device are arranged such that movement of the locking device from the first position to the second position causes the blocking device to move from the blocking position to the releasing position.

### TECHNICAL FIELD

The present disclosure generally relates to a cassette for receiving a medicament container. In particular, a cassette for receiving a medicament container, and a medicament delivery device comprising a cassette, are provided.

### BACKGROUND

A wide range of medicament delivery devices for self-administration of medicaments are known. Some medicament delivery devices have two parts interconnected for the use, such as a base unit and a cassette. The base unit usually comprises a drive mechanism, such as a motor and/or a battery, and can thus be reused for multiple medicament deliveries, such as injections. The cassette is often containing or receiving the medicament container with the medicament such as a syringe, a cartridge or the like, which then can be thrown away after the use of the medicament and substituted with a new one. One example of such medicament delivery device is an auto-injector.

In some prior art solutions, the user needs to load the cassette into the base unit, pull off a cap from the cassette, and prepare a needle (e.g. to pierce a membrane) before injection. Upon activation of the medicament delivery device, the medicament is expelled through a medicament delivery member, for example a needle or a nozzle. The activation may be made by pressing a button on the base unit or automatically by retraction of a guard.

WO 2019122946 A1 discloses an automatic injector device comprising a reusable motorized transmission assembly and a single-use disposable, drug delivery assembly. The drug delivery assembly comprises a needle, a needle guard and a device cap.

### SUMMARY

In the present disclosure, when the term “distal” is used, this refers to the direction pointing away from the dose delivery site. When the term “distal part/end” is used, this refers to the part/end of the delivery device, or the parts/ends of the members thereof, which under use of the medicament delivery device is/are located furthest away from the dose delivery site. Correspondingly, when the term “proximal” is used, this refers to the direction pointing to the dose delivery site. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially”, “rotation”, “rotational” and “rotationally” refer to a direction generally perpendicular to the longitudinal direction and at least partially extending around the longitudinal direction.

According to one aspect, there is provided a cassette for receiving a medicament container, the cassette comprising a medicament delivery member; a guard for covering the medicament delivery member; a shell movable between a proximal shell position where movement of the guard is prevented by the shell, and a distal shell position where the guard is exposed from the shell; a biasing device arranged to force the shell towards the distal shell position; a blocking device movable from a blocking position where the shell is blocked from moving to the distal shell position by means of the biasing device, to a releasing position where the shell is released; and a locking device movable from a first position to a second position in use; wherein the blocking device and the locking device are arranged such that movement of the locking device from the first position to the second position causes the blocking device to move from the blocking position to the releasing position.

By means of the shell that is movable from the proximal shell position to the distal shell position, the shell can prevent movement of the guard in the proximal shell position but does not have to be removed from the cassette prior to medicament delivery. A step of removing a cap from the cassette prior to medicament delivery according to the prior art can therefore be eliminated.

The movement of the shell from the proximal shell position to the distal shell position provides a ready to use indication to the user. When the shell adopts the distal shell position, the guard may protrude beyond the shell. This exposure of the guard indicates to the user that the cassette is unlocked and ready to perform medicament delivery. When the shell adopts the distal shell position, the shell does not prevent the guard. The shell may cover the guard in the proximal shell position.

Furthermore, the cassette can be formed with various mechanical arrangements. For example, there is provided a cassette for receiving a medicament container, the cassette comprising: a guard extending along a longitudinal axis between a proximal end and a distal end; a shell axially movable along the longitudinal axis relative to the guard; a biasing device adjacent to a proximally directed surface of the shell at one end, and adjacent to a distally directed surface of the guard at the other end; a blocking device comprising a blocking structure extending in a direction transverse to the longitudinal axis and a guide track axially and circumferentially extending in the of the longitudinal axis; wherein the blocking device is movable between a blocking position where the blocking structure is adjacent to a distally directed surface of the shell and a releasing position where the blocking structure is spaced apart to the distally directed surface of the shell in the direction of the longitudinal axis; and a locking device movable relative to the blocking device; wherein the locking device comprises a protrusion protruding into the guide track of the blocking device.

It should be noted that, the guard is designed for covering the medicament delivery member. However, the medicament delivery member is not necessary a part of the cassette. For example, the medicament delivery member can be removably attached to the cassette before an end user carrying out a medicament delivery operation with the cassette. Alternatively, the cassette comprises a medicament delivery member.

According to one example, the locking device can be moved from the first position to the second position manually by the end user, so that an unintentional access to the medicament delivery member by moving the guard can be prevented. A damage on the medicament delivery member or an injury of the end user by contacting the medicament delivery member can be thus prevented.

Alternatively, according to another example, since the distal movement of the shell is triggered by the movement of the locking device from the first position to the second position, the cassette enables this movement of the shell to be automatically performed by loading the cassette into a base unit. Thus, the cassette may be configured such that the locking device moves from the first position to the second position by insertion of the cassette into the base unit. To this end, the locking device may comprise an external engaging structure for engaging a stationary structure in the base unit. The locking device may however be moved from the first position to the second position in alternative ways, e.g. by means of an actuator in the base unit, for example, the base unit may comprise a sensor to detect whether the cassette is properly inserted into the base unit; or detect whether the cassette is the proper one for the base unit, meaning that the contained medicament is correct to be delivered by the base unit, by scanning an information attached on the cassette, e.g. scanning a bar code/QR code or detecting a RFID tag. When the base unit detects the correct cassette, the actuator, e.g. a motor driven shaft or rotating ring or a spring driven rod, of the base unit may be driven or released, to move the locking device from the first position to the second position.

In one example, the locking device is movable along the longitudinal axis relative to the blocking device from the first position to the second position.

Alternatively, in another example, the locking device is movable in a direction transverse to the longitudinal axis relative to the blocking device from the first position to the second position.

Initially, the blocking device in the blocking position prevents the biasing device from forcing the shell from the proximal shell position to the distal shell position. Once the blocking device adopts the releasing position, the blocking device no longer prevents the biasing device from forcing the shell from the proximal shell position to the distal shell position.

In use of the cassette, the cassette may be properly connected to the base unit. The biasing device, the blocking device and the locking device may constitute a shell retraction mechanism.

The cassette may further comprise a housing. In this case, the guard is partially arranged within the housing and telescopic relative to the housing along the longitudinal axis. Furthermore, the shell may comprise a second distally directed surface configured to abut a proximally directed surface of the housing. Therefore, once the shell has been moved in the distal direction of the gaud under the force of the biasing device, the abutment between the second distally directed surface of the shell and the proximally directed surface of the housing can stop the distal movement of the shell. Once the shell can no longer move in the distal direction relative to the housing, the biasing device can only bias the guard in the proximal direction. Further, the shell may be arranged to move into the housing when moving from the proximal shell position to the distal shell position.

The medicament delivery member may be a needle or nozzle. The biasing device may comprise a spring, such as a coil spring. As a possible alternative, the biasing device may comprise one or more magnets.

The cassette may further comprise a longitudinal axis. Each of a proximal direction and a distal direction may be substantially parallel with, or parallel with, the longitudinal axis. A lateral direction may be substantially perpendicular to, or perpendicular to, the longitudinal axis. The medicament delivery member, the guard, the shell, the biasing device, the blocking device and/or the locking device may be substantially concentric with, or concentric with, the longitudinal axis.

The cassette may further comprise the medicament container. The medicament container may for example be a syringe or cartridge containing medicament.

The guard may be movable from a proximal guard position where the medicament delivery member is covered by the guard, to a distal guard position where the medicament delivery member is exposed from the guard. Thus, when the shell adopts the distal shell position, the guard is allowed to move from the proximal guard position to the distal guard position. The biasing device may be arranged to force the guard towards the proximal guard position. The guard can be moved from the proximal guard position to the distal guard position against the force of the biasing device by contacting an medicament delivery site with the guard and forcing the cassette towards the medicament delivery site.

The guard comprises an orifice on the proximal end of the guard, so that the medicament delivery member can protrude from the orifice and contact a medicament delivery site of the end user. For example, the medicament delivery member is arranged within the guard and telescopically relative to the orifice of the guard.

The guard may further comprise a guard membrane sealed the orifice of the guard. In this case, the medicament delivery member may be arranged to pierce the guard membrane when the guard moves from the proximal guard position to the distal guard position. The guard membrane may be arranged to protect sterility of the medicament delivery member before use.

In the blocking position, the blocking device may be arranged to directly block the shell from moving to the distal shell position. That is, the shell may be in direct contact with the blocking device when the shell is in the proximal shell position and the blocking device is in the blocking position.

The blocking device may be arranged to rotate relative to the longitudinal axis from the blocking position to the releasing position. In this case, the blocking device may be referred to as a rotator. The rotation may be around the longitudinal axis of the cassette.

Alternatively, the blocking device may move linearly in a direction transverse to the longitudinal axis from the blocking position to the releasing position. In this case, the blocking device may be referred to as a slider.

The locking device may be movable along the longitudinal axis relative to the blocking device from the first position to the second position. The movement from the first position to the second position may be a proximal movement relative to the cassette. The movement may be parallel with the longitudinal axis of the cassette. The locking device may in this case be prevented from rotating around the longitudinal axis.

As a possible alternative variant, the locking device may be arranged to rotate from the first position to the second position and the blocking device may be arranged to move linearly from the blocking position to the releasing position.

The cassette may further comprise a transmission arranged to transmit a movement of the locking device from the first position to the second position to a movement of the blocking device from the blocking position to the releasing position.

As a possible alternative, the blocking device and the locking device may be integrally formed. In this case, the shell may comprise one or more arms and the blocking device may be arranged to bend these arms to release the shell when the blocking device moves from the blocking position to the releasing position.

The transmission may comprise a guide track and a protrusion arranged to follow the guide track. The guide track may be provided on the blocking device and the protrusion may be provided on the locking device.

The guide track may comprise an inclination such that movement of the protrusion along the inclination causes the blocking device to rotate from the blocking position to the releasing position. The inclination may be inclined, e.g. 10 degrees to 40 degrees, relative to the longitudinal axis.

The guide track may further comprise a bump. The bump may be arranged to be engaged by the protrusion such that the locking device is prevented from returning from the second position to the first position. The bump may be arranged in the inclination.

The shell may comprise a shell engaging structure. The shell engaging structure may comprise an arm or slot, such as a laterally inwardly protruding arm, a laterally arranged recess or cut-out.

The guard may comprise a guard engaging structure, such as a slot or an arm. In this case, the shell engaging structure may be arranged to travel along the guard engaging structure. When the shell engaging structure is in a proximal end of the guard engaging structure, the guard is prevented from moving distally. When the shell engaging structure has moved distally in the guard engaging structure, e.g. due to the movement of the shell from the proximal shell position to the distal shell position, the guard is allowed to move distally relative to the shell such that the shell engaging structure travels along the guard engaging structure.

The cassette may further comprise a delivery member holder holding the medicament delivery member. In this case, the delivery member holder may be arranged to be moved distally by movement of the shell from the proximal shell position to the distal shell position. The delivery member holder may be substantially concentric with, or concentric with, the longitudinal axis.

The delivery member holder may comprise a delivery member engageable structure. In this case, the shell engaging structure may be arranged to engage the delivery member engageable structure such that the delivery member holder is moved distally by movement of the shell from the proximal shell position to the distal shell position. The delivery member engageable structure may comprise an arm, such as a laterally outwardly protruding arm. The shell engaging structure may be arranged to move relative to the delivery member engageable structure prior to engaging the delivery member engageable structure.

The cassette may further comprise a container holder for holding the medicament container, the container holder comprising a holder membrane. In this case, the medicament delivery member may be arranged to pierce the holder membrane when the delivery member holder is moved distally together with the shell under a biasing force of the basing device. In this variant, the movement of the locking device from the first position to the second position triggers both retraction of the shell and preparation of the medicament delivery member. Thus, this variant further facilitates the operation for the user in comparison with prior art solutions where the user may have to manually attach a needle before having the medicament delivery operation. The container holder may be substantially concentric with, or concentric with, the longitudinal axis. The holder membrane may be arranged to protect sterility of the medicament delivery member before use.

Alternatively, or in addition, the medicament delivery member may be arranged to pierce a container membrane of the medicament container when the delivery member holder is moved distally by movement of the shell from the proximal shell position to the distal shell position.

When the shell is in the proximal shell position and the guard is in the proximal guard position, the delivery member holder and the medicament delivery member may be protected by the guard membrane and the holder membrane. Sterilization of the medicament delivery member can thereby be kept.

The medicament delivery member may first penetrate the holder membrane, and optionally also a container membrane, by distal movement of the medicament delivery member due to the distal movement of the shell. The medicament delivery member may then penetrate the guard membrane by distal movement of the guard relative to the medicament delivery member.

The delivery member holder may be connected to the container holder by means of a snap-fit. The snap-fit may be arranged to be released when the delivery member holder is moved distally by movement of the shell from the proximal shell position to the distal shell position.

The cassette may further comprise a flexible tube surrounding the medicament delivery member. In one example, the flexible tube is connected to the guard and to the delivery member holder. In this case, the flexible tube may be arranged to be unfolded by distal movement of the shell. The flexible tube may surround the medicament delivery member to form a sealingly closed volume around the same. The same closed volume may for example also be sealed by the guard membrane.

The flexible tube may be substantially concentric with, or concentric with, the longitudinal axis. The flexible tube may be made of rubber.

The flexible tube may have a varied thickness. In this case, the flexible tube may have a larger thickness in a proximal region than in a distal region. This causes the flexible tube to be more prone to fold and unfold in the distal region. The folding of the flexible tube enables the flexible tube to adapt to different distances between the guard and the delivery member holder.

The locking device may comprise a locking engageable structure. In this case, the guard may comprise a locking engaging structure arranged to move distally past the locking engageable structure and arranged to be locked in the locking engageable structure by a proximal movement following the movement distally past the locking engageable structure. This variant enables the guard to be locked in a position covering the medicament delivery member (e.g. in the proximal guard position) after completion of medicament delivery. In this way, injuries and contamination can be prevented. The locking engageable structure and the locking engaging structure may constitute a guard lockout mechanism.

The locking engaging structure may comprise a hook. The hook may be flexible. The locking engageable structure may comprise an opening for receiving the hook. The locking engageable structure may further comprise a slope. The slope may be arranged proximally of the opening. The hook and the slope may be configured such that when the hook travels distally, the hook contacts the slope and is caused to flex. The flexing in turn prevents the hook from engaging with the opening during further distal movement of the hook. However, when the hook moves in the proximal direction after having passing the opening in the distal direction, the hook eventually flexes into the opening. In this way, the guard is locked relative to the locking device.

According to a further aspect, there is provided a medicament delivery device comprising a cassette according to the present disclosure. The medicament delivery device comprises a base unit releasably attached to the cassette; wherein the base unit comprises a stationary structure; and wherein the locking device comprises an external engaging structure arranged to engage the stationary structure by insertion of the cassette into the base unit to cause the locking device to move proximally relative to the cassette from the first position to the second position.

The base unit is arranged to drive expulsion of medicament from a medicament container in the cassette when the cassette is attached to the base unit. The base unit may be a power unit, namely the base unit may comprise a power source, e.g. spring, gas canister, motor; and a force transmitter to transfer an output force from the power source to the cassette. Alternatively, the base unit may comprise a force converting device for transferring the end user's manual force to the cassette. The cassette may be disposable and the base unit may be for multiple use.

The base unit may comprise a stationary structure. In this case, the locking device may comprise an external engaging structure. The external engaging structure may be arranged to engage the stationary structure by insertion of the cassette into the base unit to cause the locking device to move proximally relative to the cassette from the first position to the second position. When the cassette is inserted distally into the base unit, the stationary structure prevents distal movement of the external engaging structure. As a consequence, the locking device moves proximal relative to the cassette.

Thus, the medicament delivery device may be configured such that the locking device moves from the first position to the second position by insertion of the cassette into the base unit. The locking device may comprise one or more engaging arms. In this case, the external engaging structure may be provided on the engaging arms.

The locking device may however be moved from the first position to the second position in alternative ways, e.g. by means of an actuator in the base unit arranged to move the external engaging structure such that the locking device moves from the first position to the second position.

The base unit may comprise a switch. In this case, the cassette may comprise a switch activation part arranged to move distally in response to movement of the guard from the proximal guard position to the distal guard position to activate the switch. The cassette may comprise a trigger member and the switch activation part may be provided on the trigger member. Alternatively, the switch activation part may be arranged on the guard.

A stroke of the guard from the proximal guard position to the distal guard position may be longer than a resulting stroke of the switch activation part.

The guard may be arranged to move relative to the switch activation part during a first part of the stroke of the guard, and the guard may be arranged to move together with the switch activation part during a second part of the stroke of the guard.

### BRIEF DESCRIPTION OF THE DRAWINGS

Further details, advantages and aspects of the present disclosure will become apparent from the following description taken in conjunction with the drawings, wherein:

FIG. 1: schematically represents a perspective view of a cassette;

FIG. 2: schematically represents a side view of a medicament delivery device comprising the cassette and a base unit;

FIG. 3: schematically represents a side view of the medicament delivery device after insertion of the cassette into the base unit;

FIG. 4: schematically represents a side view of the medicament delivery device during medicament delivery to an medicament delivery site;

FIG. 5: schematically represents a perspective exploded view of the cassette;

FIG. 6: schematically represents a cross-sectional side view of the cassette;

FIG. 7: schematically represents a further cross-sectional side view of the cassette;

FIG. 8: schematically represents a partial side view of the cassette;

FIG. 9: schematically represents a partial side view of the cassette after movement of a locking device to a second position and movement of a blocking device to a releasing position;

FIG. 10: schematically represents a cross-sectional side view of the cassette according to FIG. 9;

FIG. 11: schematically represents a cross-sectional side view of the cassette during distal movement of a shell from a proximal shell position;

FIG. 12: schematically represents a cross-sectional side view of the cassette when the shell has moved to a distal shell position;

FIG. 13: schematically represents a partial perspective side view of the cassette where a guard is in a proximal guard position;

FIG. 14: schematically represents a partial cross-sectional side view of the cassette according to FIG. 13;

FIG. 15: schematically represents a partial cross-sectional side view of the cassette when the guard has moved to a distal guard position; and

FIG. 16: schematically represents a partial cross-sectional view of the cassette after proximal movement of the guard from the distal guard position.

### DETAILED DESCRIPTION

In the following, a cassette for receiving a medicament container, and a medicament delivery device comprising a cassette, will be described. The same or similar reference numerals will be used to denote the same or similar structural features.

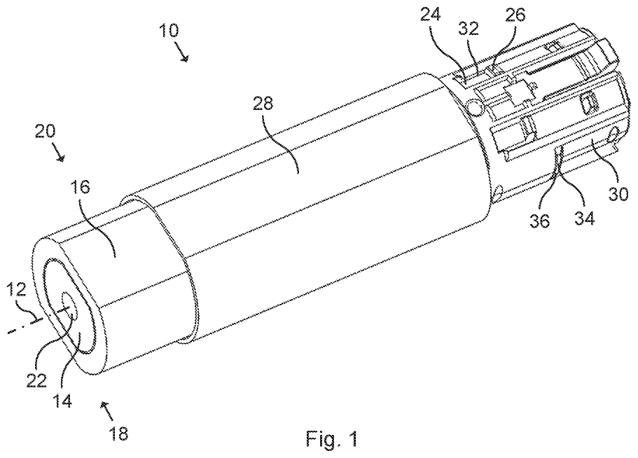

FIG. 1 schematically represents a perspective view of a cassette 10. The cassette 10 comprises a medicament delivery member (not shown), a medicament container (not shown) and a longitudinal axis 12. The cassette 10 further comprises a guard 14 and a shell 16. In FIG. 1, the guard 14 is in a proximal guard position 18 and the shell 16 is in a proximal shell position 20. The respective end surfaces of the guard 14 and the shell 16 are aligned at the proximal end of the cassette 10 (the left end in FIG. 1). The guard 14 comprises a guard membrane 22.

The cassette 10 further comprises a locking device 24. The locking device 24 comprises external engaging structures 26, here exemplified as protrusions.

The cassette 10 of this example further comprises a housing 28 and a distal cap 30. The shell 16 is positioned radially (with respect to the longitudinal axis 12) inside the housing 28. The guard 14 is positioned radially inside the shell 16.

The housing 28 comprises housing slots 32. Each housing slot 32 extends parallel with the longitudinal axis 12. Each external engaging structure 26 is arranged to travel along an associated housing slot 32.

The cassette 10 of this example further comprises two trigger members 34 (only one is visible in FIG. 1). Each trigger member 34 comprises a switch activation part 36, here exemplified as a protrusion.

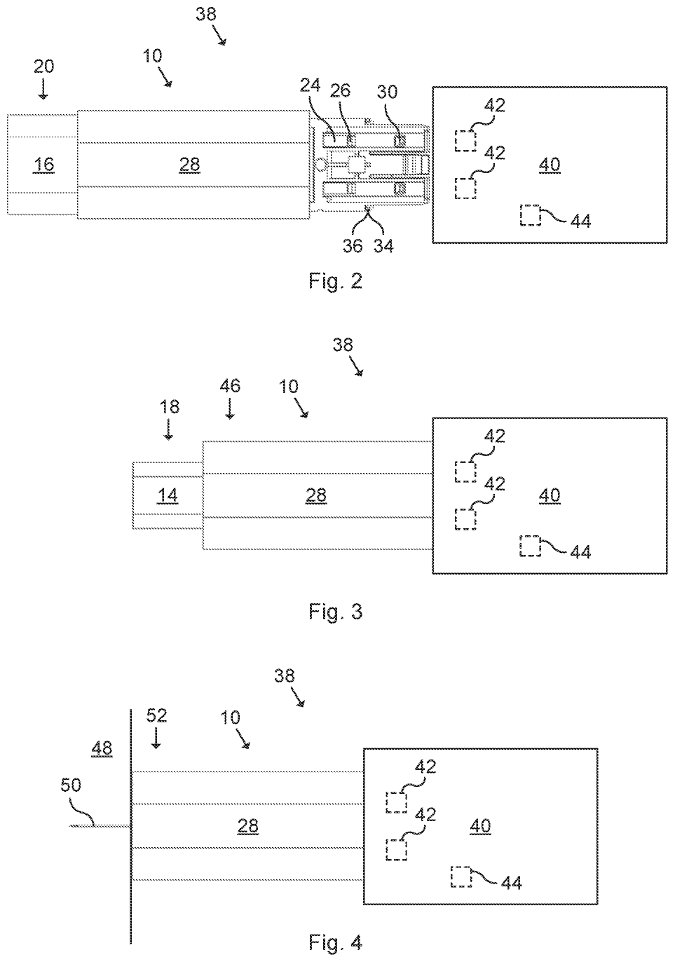

FIG. 2 schematically represents a side view of a medicament delivery device 38 comprising the cassette 10 and a base unit 40. The cassette 10 is disposable and the base unit 40 is for multiple use. The base unit 40 is arranged to drive expulsion of medicament from the medicament container in the cassette 10 when the cassette 10 is attached to the base unit 40. The base unit 40 of this example comprises stationary structures 42 and a switch 44.

Before the cassette 10 is connected to the base unit 40 (as shown in FIG. 2), neither the shell 16 not the guard 14 can be moved distally. The shell 16 is prevented from moving distally from the proximal shell position 20 by means of a blocking device (not shown) and the guard 14 is prevented from moving distally from the proximal guard position 18 by the shell 16. The user is therefore protected from a needle or other medicament delivery member inside the cassette 10. This reduces the risk of injury and contamination.

FIG. 3 schematically represents a side view of the medicament delivery device 38 after insertion of the cassette 10 into the base unit 40. The insertion of the cassette 10 into the base unit 40 constitutes a first step for the user. Once the cassette 10 is properly connected to the base unit 40, the cassette 10 may be said to be in use.

When the cassette 10 is inserted into the base unit 40, the shell 16 is retracted, i.e. moved distally from the proximal shell position 20 to a distal shell position 46. The user can therefore merely insert the cassette 10 into the base unit 40 to retract the shell 16 to expose the guard 14. The user does not need to take any dedicated action to expose the guard 14, such as removing a cap. The exposure of the guard 14 also provides a very clear ready to use indication to the user. In this example, the shell 16 is positioned entirely within the housing 28 in the distal shell position 46.

The shell 16 is retracted as a consequence of the insertion of the cassette 10 into the base unit 40. During insertion of the cassette 10 into the base unit 40, the external engaging structures 26 contact associated stationary structures 42 inside the base unit 40. Further insertion of the cassette 10 thereby causes the locking device 24 to move proximally relative to the remainder of the cassette 10. In other words, the external engaging structures 26 become stationary due to the contact with the stationary structures 42 and the cassette 10 moves distally relative to the external engaging structure 26. This proximal movement of the locking device 24 relative to the cassette 10 triggers the retraction of the shell 16, as will be described in further detail below.

Alternatively, an actuator in the base unit 40 may be driven to move the external engaging structure 26 to initiate the retraction of the shell 16. In any case, the retraction of the shell 16 may be triggered by a movement of the locking device 24 relative to the remainder of the cassette 10.

When the shell 16 has adopted the distal shell position 46, the guard 14 is exposed from the shell 16 and the shell 16 no longer prevents movement of the guard 14. In this example, the insertion of the cassette 10 into the base unit 40 is the only action the user needs to take to prepare the medicament delivery device 38.

FIG. 4 schematically represents a side view of the medicament delivery device 38 during medicament delivery to an medicament delivery site 48. In FIG. 4, the medicament delivery member 50, here exemplified as a needle, can be seen.

The user may simply bring the guard 14 into contact with the medicament delivery site 48 and push the medicament delivery device 38 against the medicament delivery site 48. This causes the guard 14 to move distally, relative to the remainder of the cassette 10, from the proximal guard position 18 to a distal guard position 52. At the same time, the medicament delivery member 50 pierces the guard membrane 22 and pierces into the medicament delivery site 48. Thus, in the distal guard position 52, the medicament delivery member 50 is exposed from the guard 14.

Moreover, the distal movement of the guard 14 from the proximal guard position 18 to the distal guard position 52 causes the switch activation parts 36 to move distally. One of the switch activation parts 36 thereby activates the switch 44 in the base unit 40. This activation of the switch 44 causes the base unit 40 to drive expulsion of medicament from the medicament container through the medicament delivery member 50 into the medicament delivery site 48.

In this example, the medicament delivery is thus triggered automatically by the distal movement of the guard 14. The medicament delivery constitutes a second step for the user.

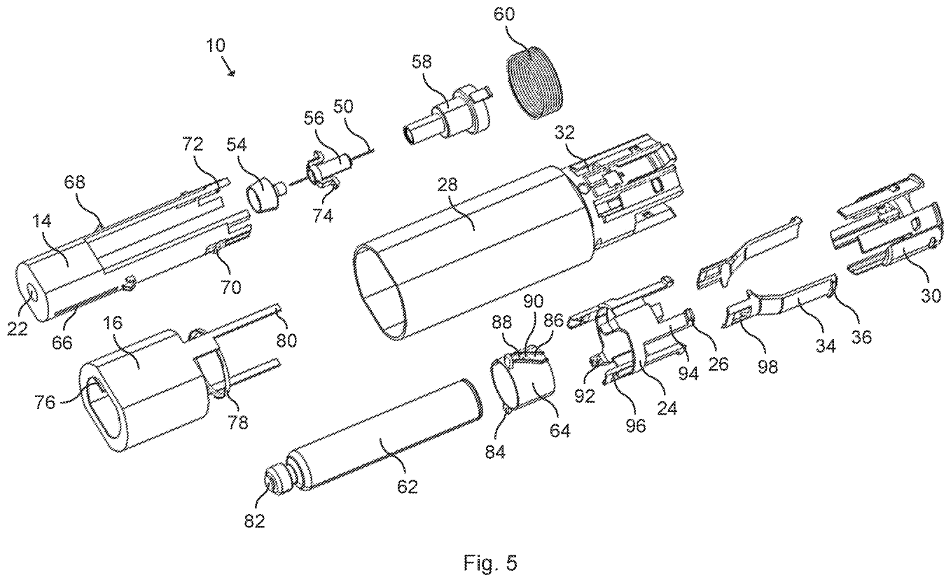

FIG. 5 schematically represents a perspective exploded view of the cassette 10. As shown in FIG. 5, the cassette 10 of this example comprises the guard 14, a flexible tube 54, a delivery member holder 56, a container holder 58, a biasing device 60, the shell 16, the housing 28, the medicament container 62, the blocking device 64, the locking device 24, the trigger members 34 and the distal cap 30.

The guard 14 comprises two guard slots 66 (only one is visible in FIG. 5). Each guard slot 66 is parallel with the longitudinal axis 12. The guard slots 66 are provided in a proximal part of the guard 14.

The guard 14 comprises a guard engaging structure, in this example the guard engaging structure is two guard slots 66 (only one is visible in FIG. 5). Each guard slot 66 is parallel with the longitudinal axis 12. The guard slots 66 are provided in a proximal part of the guard 14.

The guard 14 further comprises two locking engaging structures 70. Each locking engaging structure 70 is provided on a respective arm of the guard 14 distally of the guard biasing device supports 68.

The guard 14 further comprises two guard hooks 72. Each guard hook 72 is provided on a respective arm of the guard 14 distally of the locking engaging structures 70.

The delivery member holder 56 holds the medicament delivery member 50. The delivery member holder 56 is here exemplified as a needle hub. The delivery member holder 56 comprises two delivery member engageable structures 74. Each delivery member engageable structure 74 is here exemplified as a lateral arm extending laterally outwards from a body of the delivery member holder 56. The biasing device 60 of this example is a coil spring.

The shell 16 comprises two shell engaging structures 76, in this example is a pair of arms (only one is seen in FIG. 5). Each arm 76 extends laterally inwards from a proximal part of the shell 16.

The shell 16 further comprises a shell biasing device support 78. The shell biasing device support 78 is arranged distally of the shell engaging structures 76. The shell biasing device support 78 of this example is a ring.

The shell 16 further comprises two shell structures 80. Each shell structure 80 is here exemplified as an arm extending parallel with the longitudinal axis 12. The shell biasing device support 78 is connected to each shell structure 80.

The medicament container 62 comprises a container membrane 82. The container membrane 82 keeps the medicament inside the medicament container 62 sterile.

The blocking device 64 comprises two blocking structures 84. Each blocking structure 84 is arranged to selectively block one shell structure 80 of the shell 16.

The blocking device 64 further comprises two guide tracks 86. In this example, the blocking device 64 comprises a cylindrical body and the guide tracks 86 are oppositely arranged on the cylindrical body. Each guide track 86 comprises an inclination 88. A bump 90 is arranged in the inclination 88.

The locking device 24 comprises two protrusions 92. Each protrusion 92 is arranged to engage a corresponding guide track 86 of the blocking device 64.

The locking device 24 further comprises four engaging arms 94. Each engaging arm 94 is parallel with the longitudinal axis 12. One external engaging structure 26 is provided at a distal end of each engaging arm 94.

The locking device 24 further comprises two locking engageable structures 96. Each locking engageable structure 96 is arranged to be engaged by a corresponding locking engaging structure 70 of the guard 14.

In this example, the trigger members 34 are separate from the guard 14. As a possible alternative, the trigger members 34 may be integrally formed with the guard 14 or the switch activation part 36 may be provided on the guard 14 in other ways.

The switch activation part 36 is arranged at a distal end of each trigger member 34. Each trigger member 34 further comprises a trigger member opening 98. Each trigger member opening 98 is arranged to receive a guard hook 72 of the guard 14.

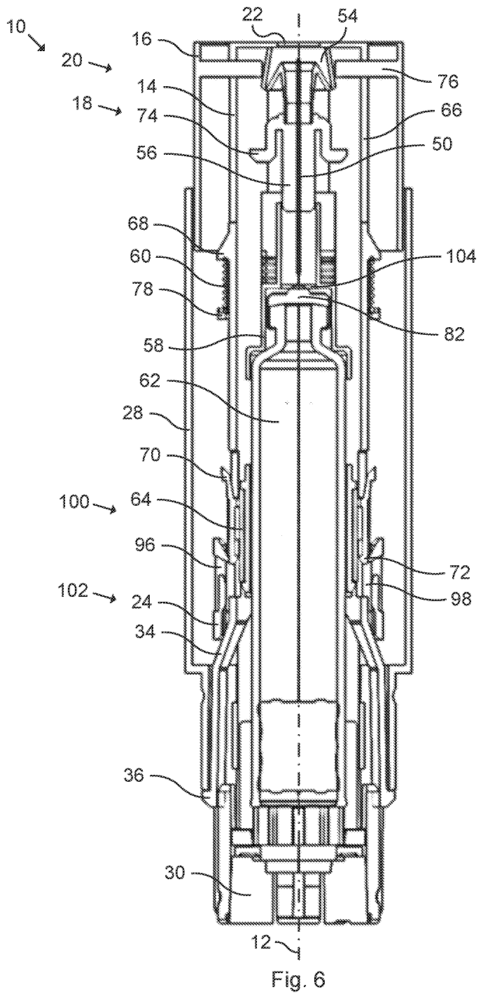

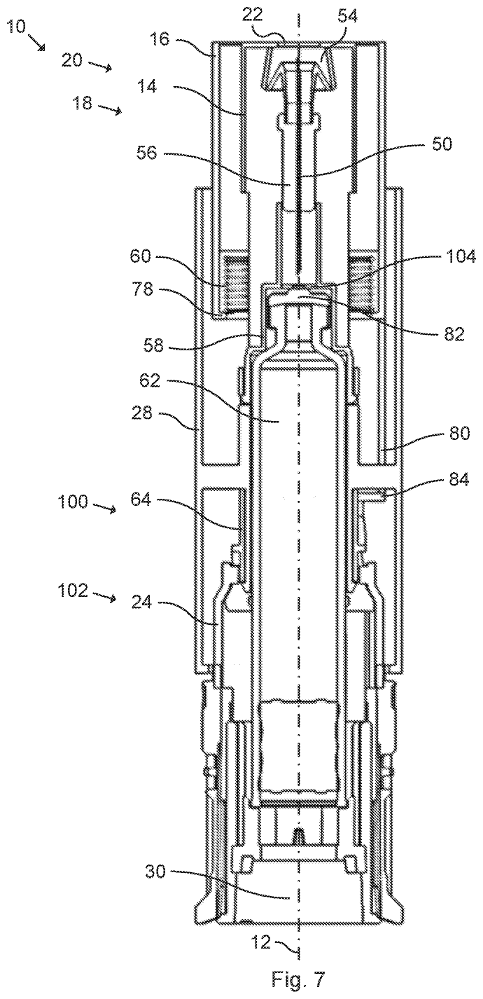

FIG. 6 schematically represents a cross-sectional side view of the cassette 10 and FIG. 7 schematically represents a further cross-sectional side view of the cassette 10. The views in FIGS. 6 and 7 are orthogonal to each other. As shown in FIGS. 6 and 7, each of the medicament delivery member 50, the guard 14, the shell 16, the biasing device 60, the blocking device 64, the locking device 24, the delivery member holder 56, the container holder 58 and the flexible tube 54 is concentric with the longitudinal axis 12.

The container holder 58 holds the medicament container 62 at a proximal end thereof. The delivery member holder 56 is connected to the container holder 58 by means of a snap-fit.

A proximal end of the flexible tube 54 is connected to the guard 14 and a distal end of the flexible tube 54 is connected to the delivery member holder 56. In FIG. 6, the flexible tube 54 is folded. The flexible tube 54 is made of rubber.

In the proximal guard position 18, the guard 14 covers the medicament delivery member 50. In FIGS. 6 and 7, the cassette 10 is in the same state as in FIG. 2. That is, the shell 16 is in the proximal shell position 20 and the guard 14 is in the proximal guard position 18.

The biasing device 60 acts between the guard biasing device support 68 and the shell biasing device support 78. In the state in FIGS. 6 and 7, the biasing device 60 is compressed. Consequently, the biasing device 60 forces the shell 16 distally from the proximal shell position 20. However, the blocking device 64 prevents the shell 16 from being moved distally by the biasing device 60.

In FIGS. 6 and 7, the blocking device 64 is in a blocking position 100 and the locking device 24 is in a first position 102. As shown in FIG. 6, the shell engaging structures 76 are in respective proximal ends of the guard slots 66. The guard 14 is therefore prevented to move distally when the shell 16 is in the proximal shell position 20.

A proximal part of the medicament delivery member 50, i.e. protruding proximally from the delivery member holder 56, is sealingly closed by the guard membrane 22 and the flexible tube 54. The container holder 58 comprises a holder membrane 104. A distal part of the medicament delivery member 50, i.e. protruding distally from the delivery member holder 56, is sealingly closed by container holder 58 and the holder membrane 104. The guard membrane 22, the flexible tube 54 and the holder membrane 104 thereby protect sterility of the medicament delivery member 50 before use.

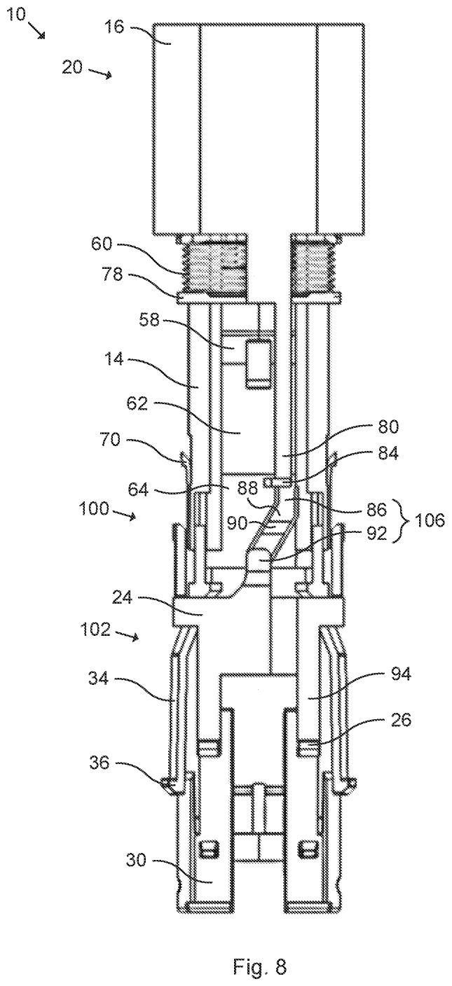

FIG. 8 schematically represents a partial side view of the cassette 10. In FIG. 8, the cassette 10 is in the same state as in FIGS. 2, 6 and 7. In the blocking position 100, the blocking device 64 blocks distal movement of the shell 16 from the proximal shell position 20. As shown in FIG. 8, the blocking structure 84 is aligned with and contacts the shell structure 80 such that distal movement of the shell 16 is stopped.

The locking device 24 is prevented from rotating around the longitudinal axis 12 due to the engagement of the external engaging structure 26 in the housing slots 32. The inclination 88 of the guide track 86 is angled approximately 30 degrees to the longitudinal axis 12. The guide track 86 and the protrusion 92 form one example of a transmission 106.

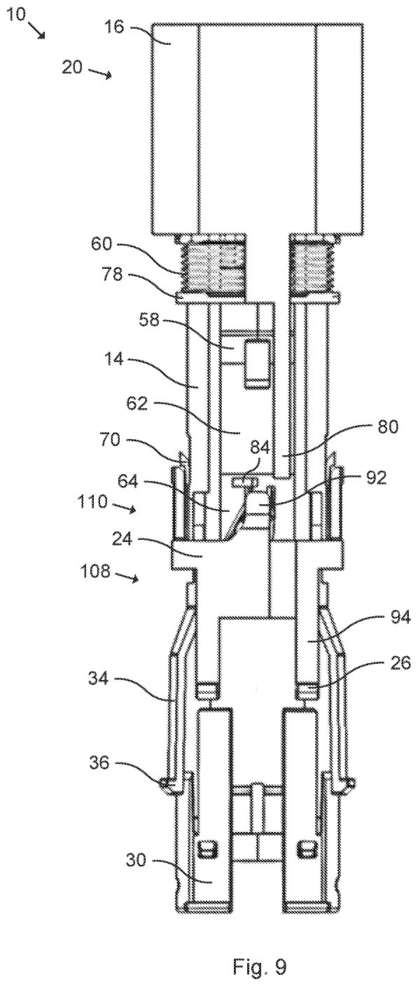

FIG. 9 schematically represents a partial side view of the cassette 10. In FIG. 9, the locking device 24 has moved linearly in the proximal direction from the first position 102 to a second position 108, e.g. by means of the engagement between the external engaging structures 26 and the stationary structures 42 in the base unit 40 as described above. The movement of the locking device 24 from the first position 102 to the second position 108 is transmitted by the transmission 106 to a rotation of the blocking device 64 about the longitudinal axis 12 from the blocking position 100 to a releasing position 110.

More specifically, the linear proximal movement of the locking device 24 causes the protrusion 92 to slide along the guide track 86. When the protrusion 92 travels along the inclination 88, the blocking device 64 is caused to rotate from the blocking position 100 to the releasing position 110. The bump 90 prevents the protrusion 92 from returning along the inclination 88. In this way, the locking device 24 is prevented from returning from the second position 108 to the first position 102.

As shown in FIG. 9, the blocking structure 84 is now offset with respect to the shell structure 80. The shell 16 is thereby released. That is, the shell 16 loses its support and the biasing device 60 thereby forces the shell 16 to move distally from the proximal shell position 20 to the distal shell position 46.

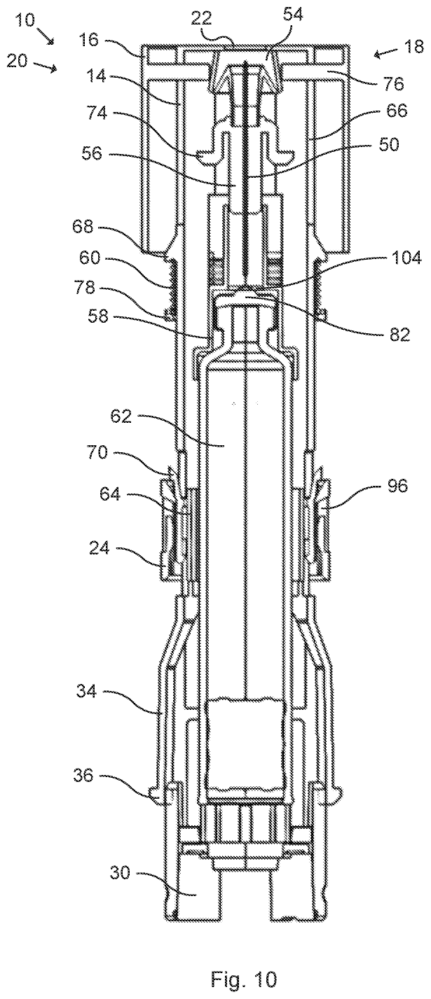

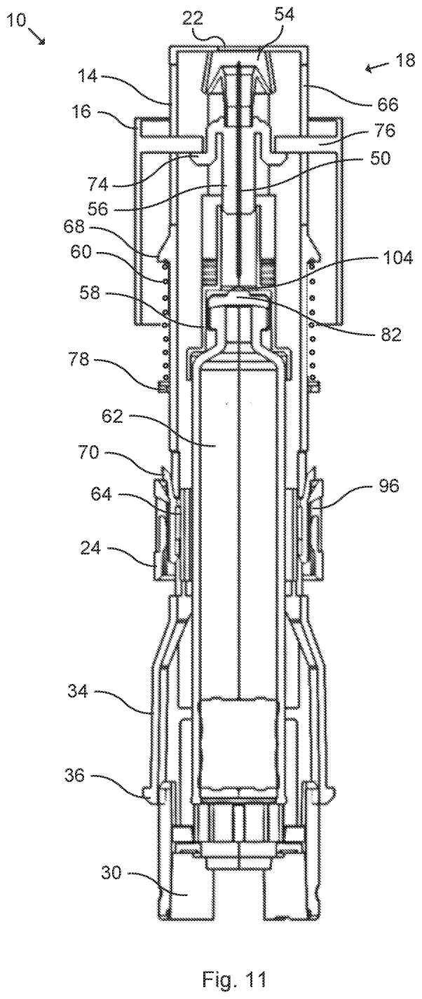

FIG. 10 schematically represents a cross-sectional side view of the cassette 10 according to FIG. 9. FIG. 11 schematically represents a cross-sectional side view of the cassette 10 during distal movement of the shell 16 from the proximal shell position 20. When the shell 16 moved distally relative to the guard 14, the shell engaging structures 76 travel in the guard slots 66.

After some distal movement of the shell 16 from the proximal shell position 20, the shell engaging structure 76 come into contact with the delivery member engageable structure 74. Further distal movement of the shell 16 causes the snap-fit between the delivery member holder 56 and the container holder 58 to be released.

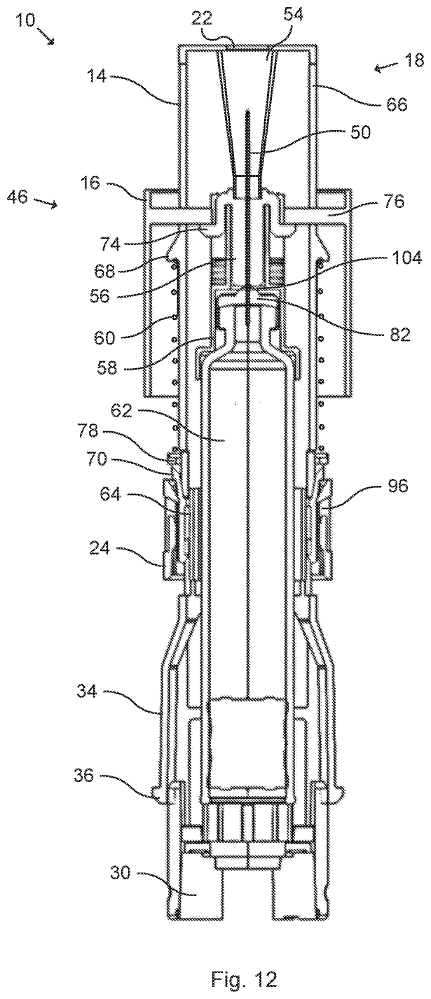

FIG. 12 schematically represents a cross-sectional side view of the cassette 10 when the shell 16 has moved to the distal shell position 46. As shown in FIG. 12, the shell engaging structures 76 are now positioned in respective distal ends of the guard slots 66. The shell 16 does therefore not prevent the guard 14. That is, the guard 14 can be pushed distally such that the shell engaging structures 76 slide along the guard slots 66.

When the shell 16 is in the distal shell position 46 and the guard 14 is in the proximal guard position 18 according to FIG. 12, the biasing device 60 forces the guard 14 into the proximal guard position 18. That is, the guard 14 can be moved distally from the proximal guard position 18 against the force of the biasing device 60.

In the distal shell position 46 shown in FIG. 12, the delivery member holder 56 has been pushed further distally due to the engagements between the shell engaging structures 76 and the delivery member engageable structures 74. As a result, the medicament delivery member 50 has pierced the holder membrane 104 and the container membrane 82 and penetrates into the medicament container 62.

When the medicament delivery member 50 has pierced the holder membrane 104 and the container membrane 82, the cassette 10 is ready to deliver medicament from the medicament container 62. The medicament delivery member 50 is thereby prepared.

The flexible tube 54 is unfolded by the distal movement of the delivery member holder 56. The flexible tube 54 thereby adapts to the changed distance between the guard 14 and the delivery member holder 56 while maintaining a lateral seal around a proximal part of the medicament delivery member 50. As shown in FIG. 12, the flexible tube 54 has a varying thickness. The flexible tube 54 is thicker in its proximal region than in its distal region.

When the cassette 10 is in the state shown in FIG. 12 and when the guard 14 is pressed against the medicament delivery site 48, the guard 14 moves distally relative to the remainder of the cassette 10 against the compression of the biasing device 60. The medicament delivery member 50 thereby pierces the guard membrane 22 and moves into the medicament delivery site 48. Thus, the user does not have to remove a cap to expose the medicament delivery member 50 prior to piercing into the medicament delivery site 48. A proximal end of the medicament delivery member 50 can therefore be kept sterile until the very moment of piercing the medicament delivery site 48. The flexible tube 54 is again folded when the delivery member holder 56 moves proximally. Medicament delivery is triggered by the distal movement of the guard 14 relative to the remainder of the cassette 10.

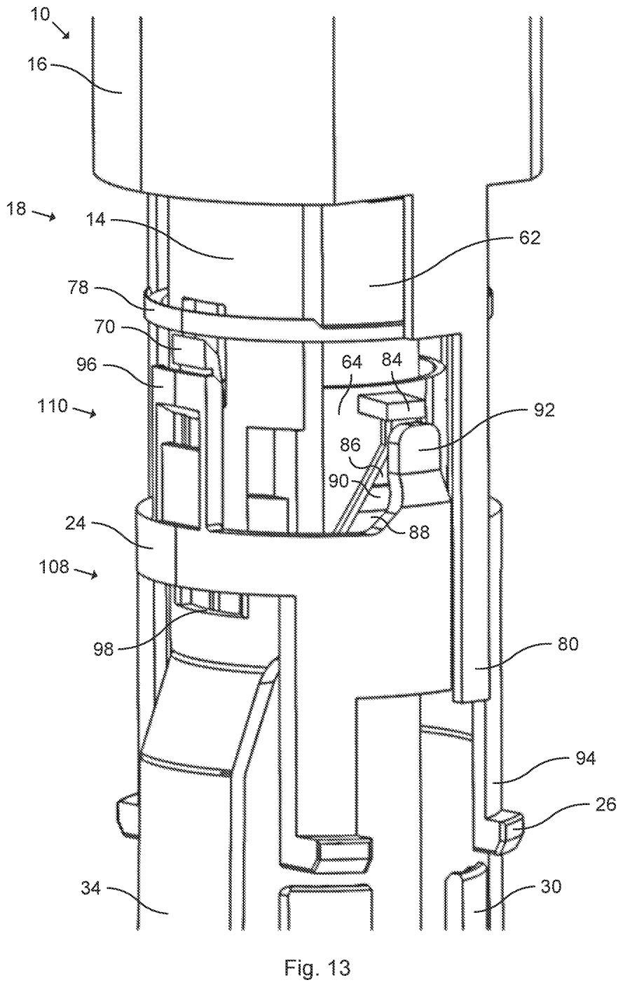

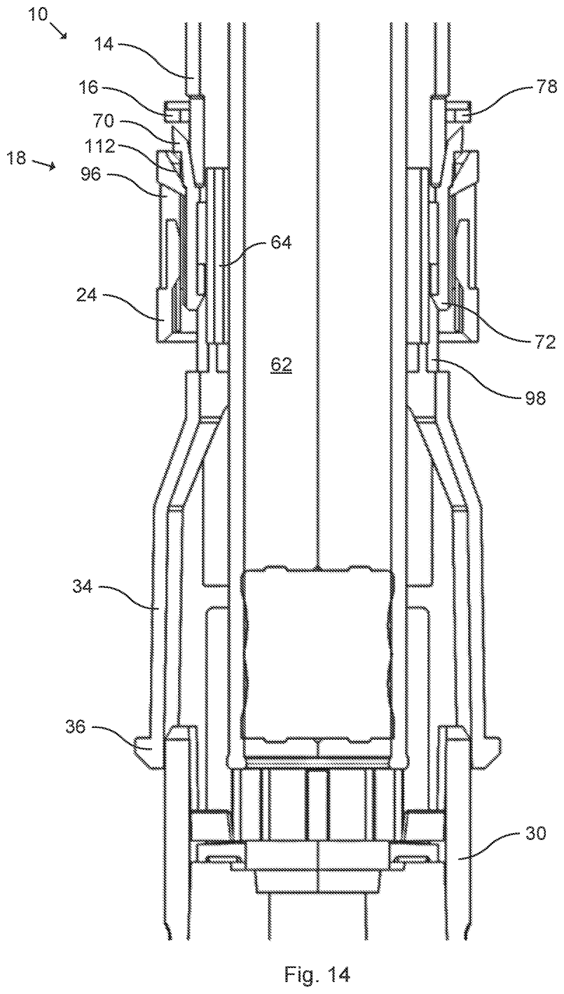

FIG. 13 schematically represents a partial perspective side view of the cassette 10 where the guard 14 is in the proximal guard position 18 and FIG. 14 schematically represents a partial cross-sectional side view of the cassette 10 according to FIG. 13. With collective reference to FIGS. 13 and 14, each locking engaging structure 70 is exemplified as a flexible hook extending laterally outwards and proximally. The locking engageable structure 96 comprises an opening and a slope 112 proximal of the opening. A distal surface of the opening is perpendicular to the longitudinal axis 12. The slope 112 extends laterally inwards and distally. The slope 112 extends laterally inside the opening of the locking engageable structure 96.

In FIG. 14, the guard hook 72 engages a proximal end of the trigger member opening 98. The guard hook 72 and the trigger member opening 98 define a maximum extended length and a maximum reduced length of the guard 14 and the trigger members 34.

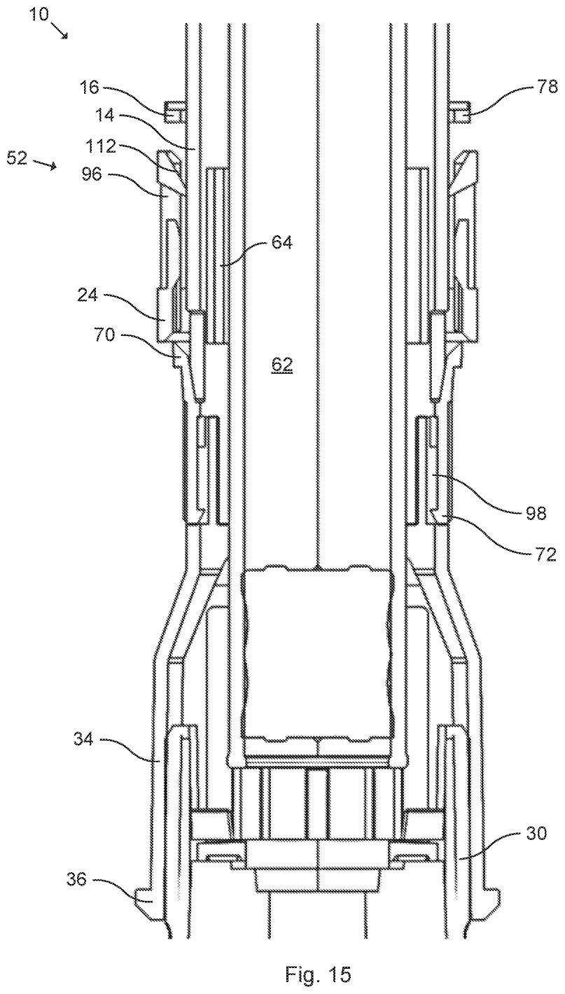

FIG. 15 schematically represents a partial cross-sectional side view of the cassette 10 when the guard 14 has moved to a distal guard position 52. When the locking engaging structure 70 moves distally from the FIG. 14 position to the FIG. 15 position, the locking engaging structure 70 engages the slope 112 and flexes laterally inwards. This causes the locking engaging structure 70 to jump over the opening of the locking engageable structure 96.

As shown in FIG. 15, the guard hook 72 engages a distal end of the trigger member opening 98. Thus, during a first part of the distal movement of the guard 14 from the proximal guard position 18, the guard 14 moves relative to the trigger members 34 which are stationary. That is, the guard hooks 72 move from a proximal end of the trigger member openings 98 to a distal end of the trigger member openings 98 during the first part of the distal movement of the guard 14.

During further movement of the guard 14 towards the distal guard position 52, the guard 14 and the trigger members 34 move together. A stroke of the guard 14 from the proximal guard position 18 to the distal guard position 52 is thereby longer than a corresponding stroke of the switch activation parts 36 provided on the trigger members 34. The stroke of the guard 14 may for example be 20 mm and the stroke of the trigger members 34 may for example be 15 mm.

The distal movement of the switch activation parts 36 activates the switch 44 in the base unit 40. The base unit 40 is thereby activated to start the medicament delivery operation. When the medicament delivery operation is completed and the cassette 10 is removed from the medicament delivery 48, the guard 14 is pushed proximally from the distal guard position 52 by means of the biasing device 60.

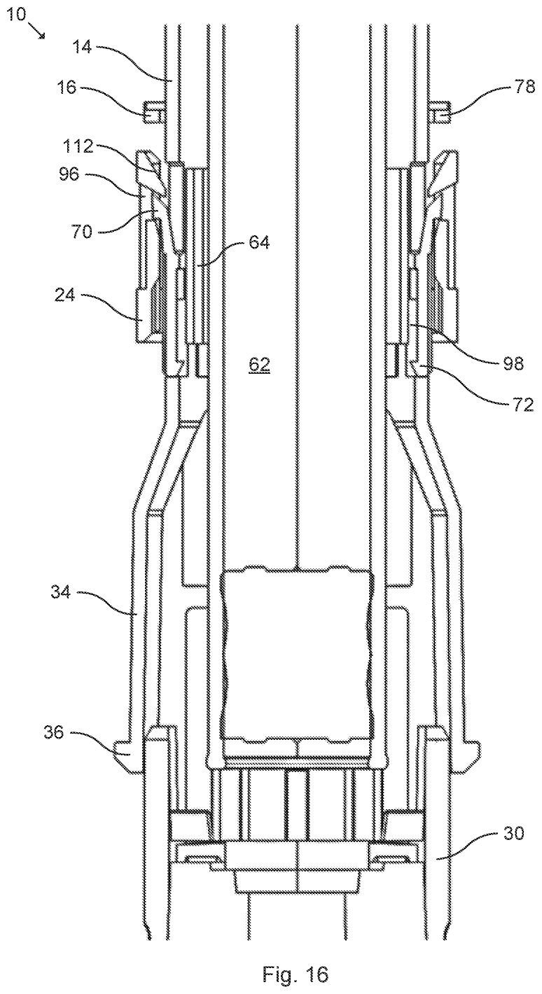

FIG. 16 schematically represents a partial cross-sectional view of the cassette 10 after proximal movement of the guard 14 from the distal guard position 52. The locking engaging structure 70 is caught and locked in the locking engageable structure 96. The distal surface of the opening of the locking engageable structure 96 prevents the guard 14 from being moved distally again. In this way, the guard 14 is locked in a state covering the medicament delivery member 50 after finishing the medicament delivery operation. This further reduces the risk of injuries and contamination.

While the present disclosure has been described with reference to exemplary embodiments, it will be appreciated that the present invention is not limited to what has been described above. For example, it will be appreciated that the dimensions of the parts may be varied as needed. Accordingly, it is intended that the present invention may be limited only by the scope of the claims appended hereto.

1-15: (canceled)

16: A cassette for receiving a medicament container, the cassette comprising:
a guard extending along a longitudinal axis between a proximal end and a distal end;
a shell axially movable along the longitudinal axis relative to the guard;
a biasing device adjacent to a proximally directed surface of the shell at one end, and adjacent to a distally directed surface of the guard at the other end;
a blocking device comprising a blocking structure extending in a direction transverse to the longitudinal axis and a guide track axially and circumferentially extending in the of the longitudinal axis; wherein the blocking device is movable between a blocking position where the blocking structure is adjacent to a distally directed surface of the shell and a releasing position where the blocking structure is spaced apart to the distally directed surface of the shell in the direction of the longitudinal axis; and
a locking device movable relative to the blocking device; wherein the locking device comprises a protrusion protruding into the guide track of the blocking device.

17: The cassette according to claim 16, wherein blocking device is rotatable relative to the longitudinal axis between the blocking position and the releasing position.

18: The cassette according to claim 16, wherein the locking device is movable along the longitudinal axis relative to the blocking device from the first position to the second position.

19: The cassette according to claim 17, wherein the guide track comprises an inclination such that movement of the protrusion along the inclination causes the blocking device to rotate from the blocking position to the releasing position.

20: The cassette according to claim 16, wherein the shell comprises a shell engaging structure; wherein the guard comprises a guard slot; and wherein the shell engaging structure is arranged to travel in the guard slot.

21: The cassette according to claim 16, comprising a medicament delivery member.

22: The cassette according to claim 21, comprising a flexible tube surrounding the medicament delivery member.

23: The cassette according to claim 21, wherein the guard comprises an orifice on the proximal end of the guard and a guard membrane sealed the orifice of the guard, wherein the medicament delivery member is arranged within the guard and telescopically relative to the orifice of the guard.

24: The cassette according to claim 21, further comprising a delivery member holder holding the medicament delivery member, wherein the delivery member holder is attached to the shell.

25: The cassette according to claim 20, wherein a delivery member holder comprises a delivery member engageable structure, and wherein the shell engaging structure is arranged to engage the delivery member engageable structure.

26: The cassette according to claim 21, further comprising a container holder for holding the medicament container, the container holder comprising a holder membrane, wherein the medicament delivery member is arranged to pierce the holder membrane when the delivery member holder is moved distally together with the shell under a biasing force of the basing device.

27: The cassette according to claim 23, wherein a flexible tube connected to the guard and to the delivery member holder, the flexible tube being arranged to be unfolded by distal movement of the shell.

28: The cassette according to claim 16, comprises a housing; wherein the guard is partially arranged within the housing and telescopic relative to the housing along the longitudinal axis.

29: The cassette according to claim 28, wherein the shell comprises a second distally directed surface configured to abut a proximally directed surface of the housing.

30: A medicament delivery device comprising a cassette according to claim 16, the medicament delivery device comprises a base unit releasably attached to the cassette; wherein the base unit comprises a stationary structure; and wherein the locking device comprises an external engaging structure arranged to engage the stationary structure by insertion of the cassette into the base unit to cause the locking device to move proximally relative to the cassette from the first position to the second position.

31: A cassette for receiving a medicament container, the cassette comprising:
a guard extending along a longitudinal axis between a proximal end and a distal end;
a shell axially movable along the longitudinal axis relative to the guard;
a spring that exerts a distal biasing force against the shell;
a blocking device comprising a blocking structure extending radially outward from an outer surface and a guide track axially and circumferentially extending along the outer surface, where the blocking device rotates between a blocking position, where the blocking structure is adjacent to a distally directed surface of the shell, and a releasing position where the blocking structure is not adjacent the distally directed surface of the shell; and
a locking device comprising an inwardly directed protrusion positioned within the guide track of the blocking device such that linear movement of the locking device in a proximal direction causes rotation of the blocking device.

32: The cassette of claim 31, wherein the spring has one end adjacent to a proximally directed surface of the shell and another end adjacent to a distally directed surface of the guard.

33: The cassette of claim 32, wherein the blocking device prevents the shell from being moved distally by the spring.

34: The cassette of claim 32, wherein movement of the locking device from a first position to a second position is transmitted by a transmission such that the blocking device rotates about the longitudinal axis from the blocking position to a releasing position.

35: The cassette of claim 32 further comprising a medicament delivery member in fluid communication with the medicament container, where a flexible tube is positioned to surround the medicament delivery member.
